(19) United States
(12) Patent Application Publication
(10) Pub. No.: US 2025/0372217 A1
(43) Pub. Date: Dec. 4, 2025

(54) SYSTEM AND METHOD FOR ACCESSING AND MANGING MEDICAL RECORD INFORMATION

(71) Applicant: EZ MED REX LLC, Destin, FL (US)

(72) Inventor: William N. Grant, III, Destin, FL (US)

(21) Appl. No.: 19/224,573

(22) Filed: May 30, 2025

Related U.S. Application Data
(60) Provisional application No. 63/653,652, filed on May 30, 2024.

(51) Int. Cl. G16H 10/60; G16H 10/40; G16H 15/00; G16H 40/67

(52) U.S. Cl. CPC G16H 10/60; G16H 10/40; G16H 15/00; G16H 40/67

(57) ABSTRACT

A system for accessing and managing medical record data is disclosed, including at least one user computing device in operable connection with a user network. An application server is in operable communication with the user network to host an application program for collecting, accessing, and managing medical record data. At least one medical record database is configured to receive a plurality of medical record data from one or more disparate medical record data source. A user interface is provided via the application program to enable the user to access the plurality of medical record data.

### CROSS-REFERENCE TO RELATED APPLICATIONS

This application claims the benefit of U.S. Provisional Patent Application No. 63/653,652, filed May 30, 2024, entitled “System and Method for Accessing and Managing Medical Record Information,” which is hereby incorporated by reference in its entirety.

### TECHNICAL FIELD

The embodiments disclosed herein generally relate to computerized systems the storage and management of medical records.

### BACKGROUND

Medical records are essential components of healthcare, documenting a patient's clinical history, diagnoses, treatments, medications, test results, and other relevant information collected throughout the continuum of care. Historically, these records were handwritten and stored in paper files within individual healthcare facilities. Each healthcare provider typically maintained its own set of records, which were often fragmented and inaccessible to other providers. This paper-based model was time-consuming to manage, vulnerable to physical damage or loss, and limited in its ability to facilitate coordinated care, especially when patients sought treatment across multiple institutions.

With the rise of digital technologies in the late 20th century, many healthcare institutions began transitioning from paper records to electronic medical record (EMR) and electronic health record (EHR) systems. These systems aimed to digitize patient data to reduce redundancy, improve accuracy, and facilitate information sharing within a single healthcare organization. By the early 2000s, government initiatives and regulations, such as the Health Information Technology for Economic and Clinical Health (HITECH) Act of 2009 in the United States, accelerated the adoption of EHR systems nationwide. These policies encouraged or required providers to implement certified EHR technology to receive incentives and avoid penalties.

Despite the proliferation of EHR systems, interoperability between them has remained a significant challenge. Many systems operate in silos and utilize proprietary data formats, hindering seamless communication between institutions. Patients are frequently required to submit formal record requests with each provider separately, often via fax or physical mail, to consolidate their own medical history. This process can take days or weeks, placing an administrative burden on both patients and providers. Moreover, even when data is transferred electronically, it may arrive in an unstructured or incomplete format, limiting its clinical utility.

Current health information exchange (HIE) platforms offer some degree of cross-provider data access but often lack user-friendly interfaces for patients and rely on provider-initiated data sharing. Patients who wish to compile and manage their own comprehensive medical records must frequently navigate multiple patient portals, download disparate file types, and manually organize the information. This decentralized and fragmented experience presents major obstacles to continuity of care, especially for individuals with complex or chronic conditions who see numerous specialists or receive treatment in multiple health systems. A more cohesive, patient-centric approach is needed to empower users with timely, secure, and complete access to their medical data across disparate systems.

### SUMMARY OF THE INVENTION

This summary is provided to introduce a variety of concepts in a simplified form that is further disclosed in the detailed description of the embodiments. This summary is not intended for determining the scope of the claimed subject matter.

The embodiments provided herein relate to a system for managing medical data records to enable a patient to view and otherwise interact with their medical records using a computing device (i.e., a smart phone, tablet, computer, or other network-enabled device). The system for accessing and managing medical record data includes at least one user computing device in operable connection with a user network. An application server is in operable communication with the user network to host an application program for collecting, accessing, and managing medical record data. At least one medical record database is configured to receive a plurality of medical record data from one or more disparate medical record data source. A user interface is provided via the application program to enable the user to access the plurality of medical record data.

The system provides users with an efficient means of accessing medical records from disparate data source. This allows users complete access to medical records, stored securely via the system.

Specifically, the embodiments enable a patient to retrieve, review, and manage their medical data via a computing device connected to a network. This architecture addresses the problem of fragmented medical records stored across multiple healthcare providers by unifying them into a single, user-friendly interface.

A core component of the invention is the user computing device, which may be a smartphone, tablet, laptop, or other internet-enabled hardware. This device communicates with the application server over a secure user network to retrieve and present the patient's medical data. By leveraging mobile and web technologies, the system ensures that users can access their records anytime and from virtually any location.

The application server plays a critical role in the invention. It hosts the application software that interfaces with external medical data sources and manages user interactions. This server authenticates users, routes data requests, formats incoming records, and provides the content to the user device in a structured, secure manner.

The medical record database is configured to store and organize a wide array of healthcare data received from various providers. These providers may include hospitals, laboratories, imaging centers, pharmacies, and outpatient clinics. The database allows for aggregation, categorization, and secure retention of a comprehensive medical history.

A user interface provided by the application software allows patients to view and interact with their health information. The interface presents data in a clear, organized fashion that supports filtering, downloading, and visualizing records. This promotes better comprehension of medical histories and enhances the user experience, particularly for those managing chronic conditions or coordinating care across multiple specialists.

The system provide the ability to aggregate records from disparate sources using a centralized logic layer. This feature eliminates the need for manual record requests and reconciliations, which are traditionally slow and error-prone. Users can access a full medical history through one application rather than logging into separate patient portals.

The system includes authentication protocols to ensure only verified users can access sensitive health information. This includes password protection, two-factor authentication, or biometric verification. These security measures are essential to meet healthcare data privacy regulations and foster user trust.

The system offers functionality for users to download their medical records in standard digital formats such as PDF, XML, or HL7-compliant files. This allows patients to store offline copies, share records with providers, or upload them to other health platforms. Empowering users with data portability supports more effective collaboration with caregivers and clinicians.

The application server is configured to perform periodic synchronization with known medical data sources. This ensures the user's database remains current with minimal effort required on the user's part. Automatic synchronization eliminates delays in data availability and increases the likelihood that clinical decisions are based on up-to-date information.

The system supports a diverse set of data types, including laboratory results, prescription histories, imaging reports, immunization records, and physician notes. By integrating these varied data forms into one platform, the invention delivers a holistic view of patient health. This is particularly valuable for complex cases where multidisciplinary care coordination is necessary.

The system is compatible with multiple operating systems and devices, including Android, iOS, Windows, and macOS platforms. Users in rural or underserved areas benefit significantly from being able to manage their healthcare remotely, without dependence on physical office visits.

An integrated alert system notifies users of newly available records, abnormal results, or critical updates. These real-time notifications help users remain proactive about their health. Timely awareness of lab results or diagnosis entries can improve treatment adherence and reduce health risks.

The medical record database is hosted in the cloud and can be accessed through secure application programming interfaces (APIs). This cloud-based design supports scalability, high availability, and seamless integration with third-party health systems. The use of APIs enables standardized data exchange and facilitates rapid deployment across diverse healthcare infrastructures.

In the method embodiment, a secure session is initiated by the user, followed by a request to the application server to retrieve health data. The server authenticates the request, pulls the data from associated provider systems, processes it, and presents it to the user. This workflow minimizes manual steps and provides a smooth, automated user experience.

Security of patient information is ensured through data encryption in transit and at rest. This includes using protocols such as SSL/TLS during network transmission and AES-256 encryption for storage. These safeguards prevent unauthorized access and support compliance with HIPAA and other relevant privacy laws.

Data categorization features automatically label and group records by source, data type, and date. This organizational structure simplifies navigation and helps users quickly identify relevant entries. Enhanced data comprehension leads to more informed decision-making and better personal health management.

The system adheres to recognized healthcare interoperability standards such as HL7 and FHIR. This compliance enables consistent data formatting and allows for easy integration with electronic health records (EHRs) and health information exchanges (HIEs). The result is greater adoption potential and improved data flow across the healthcare ecosystem.

Graphical visualization tools embedded in the user interface include timelines, bar graphs, and trend plots. These tools enable users to monitor changes in laboratory results, track medication history, or identify anomalies. Visual representation of data enhances understanding and encourages deeper engagement with health information.

Customization options allow users to define preferences for how data is displayed or filtered. For example, users can choose to highlight recent activity, show only critical results, or group records by medical provider. Personalization increases the utility and relevance of the information being presented.

A non-transitory computer-readable medium embodiment is provided, which stores instructions for performing the system operations. These instructions enable processing units to execute tasks such as authentication, record retrieval, data integration, and interface rendering. This software-based configuration supports flexible deployment across consumer and enterprise computing environments.

A sharing function is available to enable users to grant temporary access to healthcare professionals or caregivers. This feature may include the generation of a time-limited access key or the ability to export select records. Temporary access options make it easy to provide emergency information while maintaining overall data security.

The system optionally includes an audit logging feature that tracks user interactions, data retrievals, and record exports. These logs support compliance audits and can help detect suspicious activity. Maintaining a verifiable trail of access enhances transparency and accountability within the system.

Modularity is supported through the use of plug-in or add-on components. Healthcare providers or developers may create and install specialized tools, such as chronic disease management modules or wearable data integrations. This modular architecture allows the platform to evolve with emerging technologies and user needs.

The system is suitable for deployment in a wide range of technical environments, including enterprise-level infrastructure, hospital systems, and commercial cloud platforms. It may also be offered as a software-as-a-service (SaaS) solution for direct patient use. These flexible deployment options expand marketability and adaptability to healthcare provider capabilities.

In summary, the disclosed invention provides a comprehensive, secure, and user-centric solution for accessing and managing medical records. By combining data aggregation, real-time updates, customizable interfaces, and secure sharing features, the system addresses critical shortcomings in the current healthcare data landscape. The invention empowers patients with control over their health information, supports more informed care decisions, and facilitates improved clinical outcomes.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a system architecture diagram of the network infrastructure, according to some embodiments.

FIG. 2 illustrates a block diagram of the application program in operable communication with the computing system, according to some embodiments.

FIG. 3 illustrates a flowchart of a method for accessing and managing medical record data, according to some embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing in detail exemplary embodiments, it is noted that the embodiments reside primarily in combinations of components related to particular devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

In general, the embodiments provided herein relate to a system and method for managing medical record data by consolidating disparate records into a unified platform accessible via a network-connected computing device. Traditional methods for managing medical records often involve separate systems that are incompatible or isolated from one another. As a result, patients must contact multiple providers individually to obtain their full medical history, which leads to inefficiencies and increased risk of incomplete care. The disclosed invention addresses these problems by enabling centralized access, management, and retrieval of health data. By integrating multiple system components, the invention facilitates improved data interoperability and user control.

The embodiments include a user computing device, which may be a smartphone, tablet, desktop, or laptop computer configured to run a client-side application. This device serves as the primary interface between the user and the system. Through the application, users can submit data requests, review health information, and interact with alerts or summaries. The system is designed to operate across various device types and operating systems, ensuring compatibility for users with diverse technological capabilities. This flexibility increases accessibility and promotes widespread adoption.

The invention further comprises an application server that communicates with both the user computing device and external medical data sources. The server authenticates user sessions, processes inbound and outbound requests, and serves as the central logic layer that orchestrates system activity. It is also responsible for managing background processes such as data synchronization, encryption, and user notification logic. The server ensures that user requests are handled securely and efficiently, while maintaining communication with the medical record database and other system components. This centralization supports rapid scaling and efficient system resource management.

Another inventive component is the medical record database, which stores patient data aggregated from multiple external systems. The database may reside on a local server, cloud-based infrastructure, or distributed computing environment. It supports structured and unstructured data types, allowing storage of clinical notes, imaging results, laboratory data, and other healthcare-related documents. The system includes tools for indexing, sorting, and querying data to support fast retrieval and meaningful organization. This database is a key enabler of the system's data unification capability.

The system's user interface presents the collected data to the patient in an intuitive and customizable format. The interface is designed with accessibility and clarity in mind, allowing patients to navigate complex data without requiring medical or technical expertise. It supports filtering options, categorized views, and downloadable record sets. Users may also receive notifications of newly available or time-sensitive data through this interface. This promotes proactive health management and improves patient engagement with their personal healthcare.

A key feature of the system is its ability to collect and normalize data from disparate sources. These sources may include EHR systems, diagnostic laboratories, imaging centers, and pharmacy records. The system employs standard data protocols such as HL7 or FHIR to interpret and integrate the received information. The result is a cohesive and readable set of records that are consistent in formatting and classification. This process eliminates the traditional barriers to interoperability between legacy health IT systems.

Security is an essential inventive feature of the system, which uses a combination of encryption, secure authentication, and role-based access controls. Data is encrypted both at rest and during transmission to ensure compliance with applicable regulations such as HIPAA. Authentication methods may include passwords, biometrics, or multi-factor authentication depending on system configuration. Audit logging is implemented to track system access and user activity. These security measures protect patient privacy and data integrity throughout the lifecycle of system usage.

The system also includes a synchronization mechanism that periodically polls external medical record sources for new or updated data. This feature reduces the need for users to initiate manual data refreshes. Synchronization parameters may be configured based on user preferences or system policies, such as daily updates or event-triggered queries. Once new data is retrieved, it is automatically processed and integrated into the existing database. This ensures that users always have access to the most up-to-date information available.

Another functionality provided by the system is the ability for users to export, share, or print their medical records. These exports may be limited to specific date ranges, types of data, or provider sources. Files can be downloaded in secure and standardized formats that are compatible with third-party applications. In certain configurations, temporary access links may be generated for limited-time sharing with caregivers or emergency responders. This functionality empowers patients to take control of their healthcare data and supports continuity of care.

The system architecture is designed for extensibility and modularity. Developers or healthcare organizations may integrate third-party plug-ins or add-ons to extend system capabilities. For example, modules may be introduced to enable advanced analytics, chronic disease tracking, or wearable device integration. This flexibility allows the system to evolve over time and adapt to emerging healthcare technologies and user needs. The modular structure also supports upgrades and version control without disrupting core functionality.

The invention further includes functionality for cross-platform application support, with the system being deployed across both Android and iOS devices. This multi-device compatibility ensures consistent access to medical records across popular mobile platforms. Users benefit from a seamless experience, regardless of their device ecosystem. Platform-specific optimization ensures efficient resource usage and responsive interface performance.

As part of its mobile implementation, the system features a secure sign-up and OTP (One-Time Password) verification process. This two-step authentication mechanism adds a layer of protection and mitigates unauthorized access. The system has been thoroughly tested to validate the robustness of the OTP feature, and identified defects were addressed through iterative QA cycles. This ensures the integrity of user access while maintaining ease of use during registration. The dual-layered approach supports both patient privacy and regulatory compliance.

An important technical enhancement includes the integration of RESTful APIs or GraphQL interfaces as replacements for outdated web service technologies like nuSOAP. This migration improves the scalability, maintainability, and security of the platform. RESTful architecture supports asynchronous communication and enables flexible interactions between system modules and third-party health systems. These improvements align the system with modern software development standards and promote interoperability with external providers and health data networks.

The iOS mobile app, originally developed in Objective-C, is being refactored in Swift to align with Apple's modern development practices. Swift offers enhanced memory management, performance improvements, and compatibility with current iOS versions. This update improves the responsiveness and reliability of the app, particularly on devices running iOS 15 and later. Migrating the codebase also facilitates integration with Apple's health APIs and features such as Face ID authentication. This modernization ensures long-term platform support and better user experiences.

The system also addresses previous issues with UI and UX consistency across platforms. A modernized UI has been implemented to ensure alignment with industry best practices, including improved navigation flows, consistent styling, and responsive layouts. These enhancements were informed by diagnostic feedback highlighting the need for a more intuitive interface. The updated design improves accessibility and lowers cognitive burden for users navigating large medical data sets. Design principles are applied consistently across mobile and web versions to ensure uniform interaction paradigms.

Performance enhancements have been made to address loading delays and inefficient network utilization. The system now leverages optimized request handling for web services, reducing load times and server strain. Data is cached and indexed more efficiently, which accelerates rendering and response generation. Diagnostic testing confirmed significant improvements in speed and stability across both platforms. These refinements contribute to an overall smoother experience, particularly during high-traffic usage scenarios.

A modular architecture has been introduced to improve scalability and support future updates. This architecture separates the system into discrete components such as authentication, data retrieval, UI rendering, and analytics modules. This modularity allows developers to update or extend functionality without affecting the entire application. It also promotes clean code practices and enables testing of individual modules during quality assurance cycles. The system is designed with plug-and-play flexibility to incorporate future innovations or third-party integrations.

To improve maintainability, the codebase has been restructured using modern naming conventions and consistent version control. Prior iterations lacked formal Git versioning, but the updated system employs a full version-tracked repository. This enables collaborative development, rollback capability, and audit trails for system changes. It also simplifies onboarding for new development team members and supports agile deployment cycles. Formal code documentation and modular design support long-term system sustainability.

Security and compliance measures have been significantly upgraded, addressing previous vulnerabilities identified during diagnostics. These include implementation of full encryption for both stored and transmitted data, secure user session handling, and robust input validation. Authentication protocols have been enhanced to include optional multi-factor authentication. The system is being validated for compliance with data privacy regulations such as HIPAA and GDPR. These improvements ensure protection of patient information and instill confidence in users and institutional partners.

The invention now includes a QA testing framework that supports both manual and automated regression testing. The framework ensures that each release maintains core system functionality and does not introduce performance regressions. Test coverage includes sign-up, OTP, record access, data download, and notification features. Repeated test cycles by independent testers help verify bug fixes and validate stability. This systematic QA approach provides a reliable foundation for system scaling and continuous improvement.

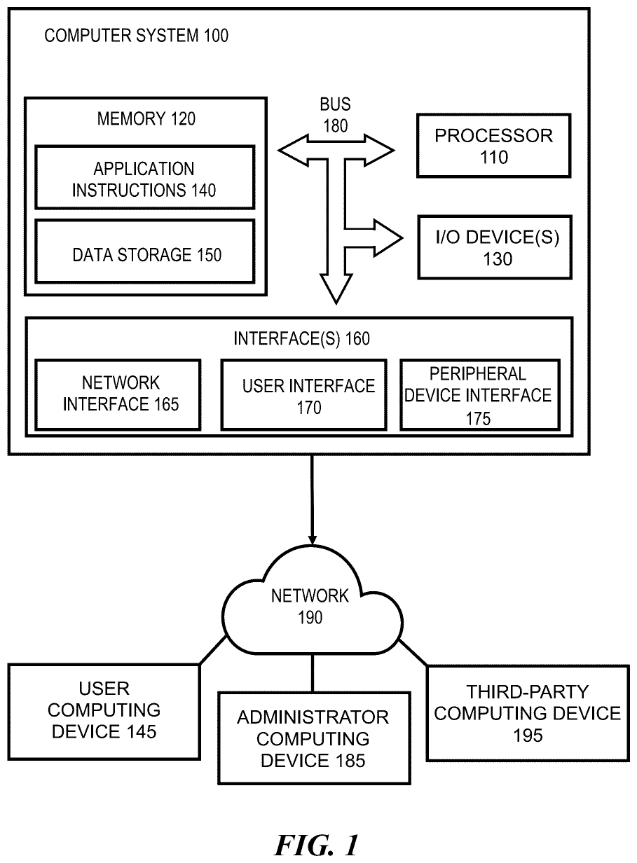

FIG. 1 illustrates an example of a computer system 100 that may be utilized to execute various procedures, including the processes described herein. The computer system 100 comprises a standalone computer or mobile computing device, a mainframe computer system, a workstation, a network computer, a desktop computer, a laptop, or the like. The computing device 100 can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

In some embodiments, the computer system 100 includes one or more processors 110 coupled to a memory 120 through a system bus 180 that couples various system components, such as an input/output (I/O) devices 130, to the processors 110. The bus 180 may be any of several types of bus structures including a memory bus or memory controller, a peripheral bus, and a local bus using any of a variety of bus architectures. For example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

Processors 110 suitable for the execution of computer readable program instructions include both general and special purpose microprocessors and any one or more processors of any digital computing device. For example, each processor 110 may be a single processing unit or a number of processing units and may include single or multiple computing units or multiple processing cores. The processor(s) 110 can be implemented as one or more microprocessors, microcomputers, microcontrollers, digital signal processors, central processing units, state machines, logic circuitries, and/or any devices that manipulate signals based on operational instructions. For example, the processor(s) 110 may be one or more hardware processors and/or logic circuits of any suitable type specifically programmed or configured to execute the algorithms and processes described herein. The processor(s) 110 can be configured to fetch and execute computer readable program instructions stored in the computer-readable media, which can program the processor(s) 110 to perform the functions described herein.

In this disclosure, the term “processor” can refer to substantially any computing processing unit or device, including single-core processors, single-processors with software multithreading execution capability, multi-core processors, multi-core processors with software multithreading execution capability, multi-core processors with hardware multithread technology, parallel platforms, and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures, such as molecular and quantum-dot based transistors, switches, and gates, to optimize space usage or enhance performance of user equipment. A processor can also be implemented as a combination of computing processing units.

In some embodiments, the memory 120 includes computer-readable application instructions 150, configured to implement certain embodiments described herein, and a database 150, comprising various data accessible by the application instructions 140. In some embodiments, the application instructions 140 include software elements corresponding to one or more of the various embodiments described herein. For example, application instructions 140 may be implemented in various embodiments using any desired programming language, scripting language, or combination of programming and/or scripting languages (e.g., Android, C, C++, C#, JAVA, JAVASCRIPT, PERL, etc.).

In this disclosure, terms “store,” “storage,” “data store,” data storage,” “database,” and substantially any other information storage component relevant to operation and functionality of a component are utilized to refer to “memory components,” which are entities embodied in a “memory,” or components comprising a memory. Those skilled in the art would appreciate that the memory and/or memory components described herein can be volatile memory, nonvolatile memory, or both volatile and nonvolatile memory. Nonvolatile memory can include, for example, read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include, for example, RAM, which can act as external cache memory. The memory and/or memory components of the systems or computer-implemented methods can include the foregoing or other suitable types of memory.

Generally, a computing device will also include, or be operatively coupled to receive data from or transfer data to, or both, one or more mass data storage devices; however, a computing device need not have such devices. The computer readable storage medium (or media) can be a tangible device that can retain and store instructions for use by an instruction execution device. The computer readable storage medium can be, for example, an electronic storage device, a magnetic storage device, an optical storage device, an electromagnetic storage device, a semiconductor storage device, or any suitable combination of the foregoing. A non-exhaustive list of more specific examples of the computer readable storage medium can include: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. In this disclosure, a computer readable storage medium is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In some embodiments, the steps and actions of the application instructions 140 described herein are embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

In some embodiments, the application instructions 140 for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The application instructions 140 can execute entirely on the user's computer, partly on the user's computer, as a stand-alone software package, partly on the user's computer and partly on a remote computer, or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

In some embodiments, the application instructions 140 can be downloaded to a computing/processing device from a computer readable storage medium, or to an external computer or external storage device via a network 190. A network adapter card or network interface in each computing/processing device receives computer readable program instructions from the network and forwards the computer readable application instructions 140 for storage in a computer readable storage medium within the respective computing/processing device.

In some embodiments, the computer system 100 includes one or more interfaces 160 that allow the computer system 100 to interact with other systems, devices, or computing environments. In some embodiments, the computer system 100 comprises a network interface 165 to communicate with a network 190. In some embodiments, the network interface 165 is configured to allow data to be exchanged between the computer system 100 and other devices attached to the network 190, such as other computer systems, or between nodes of the computer system 100. In various embodiments, the network interface 165 may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol. Other interfaces include the user interface 170 and the peripheral device interface 175.

In some embodiments, the network 190 corresponds to a local area network (LAN), wide area network (WAN), the Internet, a direct peer-to-peer network (e.g., device to device Wi-Fi, Bluetooth, etc.), and/or an indirect peer-to-peer network (e.g., devices communicating through a server, router, or other network device). The network 190 can comprise copper transmission cables, optical transmission fibers, wireless transmission, routers, firewalls, switches, gateway computers and/or edge servers. The network 190 can represent a single network or multiple networks. In some embodiments, the network 190 used by the various devices of the computer system 100 is selected based on the proximity of the devices to one another or some other factor. For example, when a first user device and second user device are near each other (e.g., within a threshold distance, within direct communication range, etc.), the first user device may exchange data using a direct peer-to-peer network. But when the first user device and the second user device are not near each other, the first user device and the second user device may exchange data using a peer-to-peer network (e.g., the Internet). The Internet refers to the specific collection of networks and routers communicating using an Internet Protocol (“IP”) including higher level protocols, such as Transmission Control Protocol/Internet Protocol (“TCP/IP”) or the Uniform Datagram Packet/Internet Protocol (“UDP/IP”).

Any connection between the components of the system may be associated with a computer-readable medium. For example, if software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. As used herein, the terms “disk” and “disc” include compact disc (CD), laser disc, optical disc, digital versatile disc (DVD), floppy disk, and Blu-ray disc; in which “disks” usually reproduce data magnetically, and “discs” usually reproduce data optically with lasers. Combinations of the above should also be included within the scope of computer-readable media. In some embodiments, the computer-readable media includes volatile and nonvolatile memory and/or removable and non-removable media implemented in any type of technology for storage of information, such as computer-readable instructions, data structures, program modules, or other data. Such computer-readable media may include RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. Depending on the configuration of the computing device, the computer-readable media may be a type of computer-readable storage media and/or a tangible non-transitory media to the extent that when mentioned, non-transitory computer-readable media exclude media such as energy, carrier signals, electromagnetic waves, and signals per se.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

In some embodiments, the system can also be implemented in cloud computing environments. In this context, “cloud computing” refers to a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

As used herein, the term “add-on” (or “plug-in”) refers to computing instructions configured to extend the functionality of a computer program, where the add-on is developed specifically for the computer program. The term “add-on data” refers to data included with, generated by, or organized by an add-on. Computer programs can include computing instructions, or an application programming interface (API) configured for communication between the computer program and an add-on. For example, a computer program can be configured to look in a specific directory for add-ons developed for the specific computer program. To add an add-on to a computer program, for example, a user can download the add-on from a website and install the add-on in an appropriate directory on the user's computer.

In some embodiments, the computer system 100 may include a user computing device 145, an administrator computing device 185 and a third-party computing device 195 each in communication via the network 190. The administrator computing device 185 is utilized by an administrative user to moderate content and to perform other administrative functions. The third-party computing device 195 may be utilized by third parties to receive communications from the user computing device, transmit communications to the user via the network, and otherwise interact with the various functionalities of the system.

Those skilled in the art would understand that each block of the flowchart illustrations and/or block diagrams, and combinations of blocks in the flowchart illustrations and/or block diagrams, can be implemented by computer readable program instructions. The computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions or acts specified in the flowchart and/or block diagram block or blocks. The computer readable program instructions can be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can be loaded onto a computer, other programmable data processing apparatus, or other device to cause a series of operational acts to be performed on the computer, other programmable apparatus, or other device to produce a computer implemented process, such that the instructions that execute on the computer, other programmable apparatus, or other device implement the functions or acts specified in the flowchart and/or block diagram block or blocks.

In this disclosure, the block diagrams in the Figures illustrate the architecture, functionality, and operation of possible implementations of systems, methods, and computer program products according to the various embodiments. Each block in the flowchart or block diagrams can represent a module, segment, or portion of instructions, which comprises one or more executable instructions for implementing the specified logical function(s). In some embodiments, the functions noted in the blocks can occur out of the order noted in the Figures. For example, two blocks shown in succession can, in fact, be executed concurrently or substantially concurrently, or the blocks can sometimes be executed in the reverse order, depending upon the functionality involved. In some embodiments, each block of the block diagrams and/or flowchart illustration, and combinations of blocks in the block diagrams and/or flowchart illustration, can be implemented by a special purpose hardware-based system that performs the specified functions or acts or carry out combinations of special purpose hardware and computer instructions.

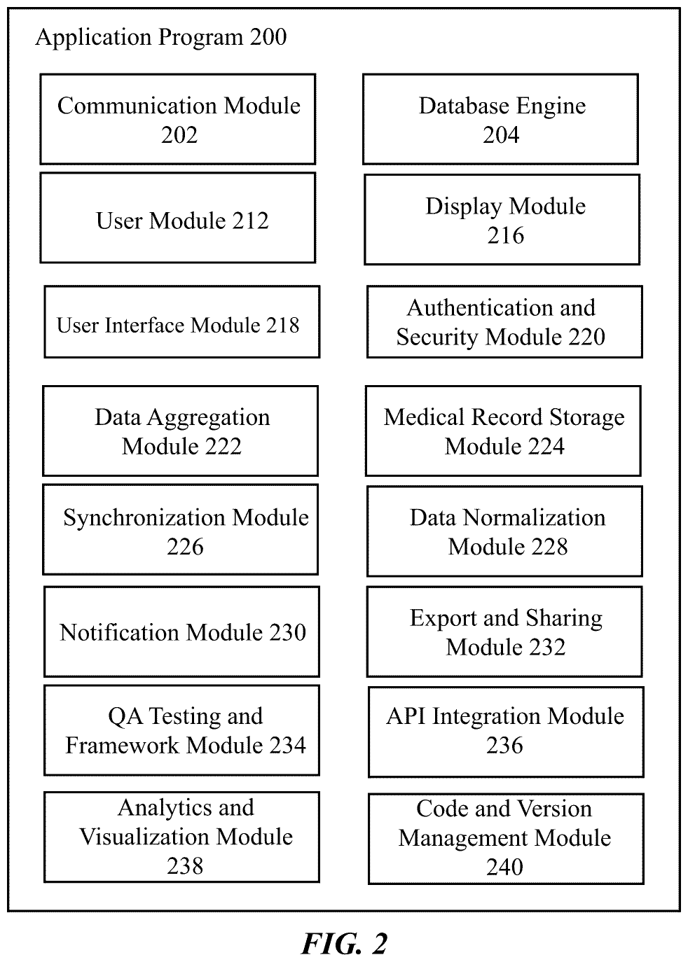

FIG. 2 illustrates an example computer architecture for the application program 200 operated via the computing system 100. The computer system 100 comprises several modules and engines configured to execute the functionalities of the application program 200, and a database engine 204 configured to facilitate how data is stored and managed in one or more databases. In particular, FIG. 2 is a block diagram showing the modules and engines needed to perform specific tasks within the application program 200.

Referring to FIG. 2, the computing system 100 operating the application program 200 comprises one or more modules having the necessary routines and data structures for performing specific tasks, and one or more engines configured to determine how the platform manages and manipulates data. In some embodiments, the application program 200 comprises one or more of a communication module 202, a database engine 204, a user module 212, a display module 216, a user interface module 218, an authentication and security module 220, a data aggregation module 222, a medical record storage module 224, a synchronization module 226, a data normalization module 228, a notification module 230, an export and sharing module 232, a QA testing and framework module 234, API integration module 236, an analytics and visualization module 238, and a code and version management module 240.

In some embodiments, the communication module 202 is configured for receiving, processing, and transmitting a user command and/or one or more data streams. In such embodiments, the communication module 202 performs communication functions between various devices, including the user computing device 145, the administrator computing device 185, and a third-party computing device 195. In some embodiments, the communication module 202 is configured to allow one or more users of the system, including a third-party, to communicate with one another. In some embodiments, the communications module 202 is configured to maintain one or more communication sessions with one or more servers, the administrative computing device 185, and/or one or more third-party computing device(s) 195. In some embodiments, the communication module 202 allows each user to transmit and receive information which may be used by the system.

The communication module 202 is configured to manage all inbound and outbound data transactions between the computing system and external entities, including healthcare providers, laboratory systems, EHR platforms, and diagnostic services. It establishes secure connections using industry-standard protocols such as HTTPS, TLS, and OAuth-based authentication to ensure the safe exchange of medical data. The module also supports both synchronous and asynchronous communication workflows, facilitating real-time queries as well as batch data retrieval.

In some embodiments, the communication module 202 includes a connection manager that tracks session states, monitors data packet integrity, and retries transmissions in the event of network interruptions. The module may also incorporate API endpoint handling logic to differentiate and prioritize requests based on source, urgency, or data type. This intelligent routing allows for efficient resource usage and ensures that the system can scale to support concurrent user sessions and high-frequency data exchanges.

In some embodiments, a database engine 204 is configured to facilitate the storage, management, and retrieval of data to and from one or more storage mediums, such as the one or more internal databases described herein. In some embodiments, the database engine 204 is coupled to an external storage system. In some embodiments, the database engine 204 is configured to apply changes to one or more databases. In some embodiments, the database engine 204 comprises a search engine component for searching through thousands of data sources stored in different locations. The database engine 204 allows each user and module associated with the system to transmit and receive information stored in various databases.

The database engine 204 is responsible for managing the storage, indexing, and retrieval of medical data within the system. It interacts directly with the medical record storage module 224 and supports structured data models optimized for querying patient histories, time-stamped events, and source-specific metadata. The engine is designed to work with SQL and NoSQL database systems depending on the deployment environment, providing both relational integrity and flexible document-based storage.

The database engine 204 also includes caching mechanisms and query optimization features to accelerate access times for frequently retrieved data. It maintains a robust schema that supports versioning of records, audit trails, and linkage across disparate sources. Additionally, the engine can be configured to implement data lifecycle policies, such as automatic archival or deletion of outdated records, ensuring compliance with retention regulations and optimizing storage capacity.

The user module 212 is designed to manage all user-related functions, including account creation, role designation, and user preferences. It supports various user types such as patients, administrative staff, and authorized third-party viewers, each with a different permission set. The module integrates with the authentication and security module 220 to enforce access controls and to store encrypted credentials and user metadata.

This module also handles session tracking, notification preferences, and interface customization options. In some embodiments, the user module 212 enables personalization of dashboard elements, medical data filters, and language settings. The module ensures that users have a seamless and secure experience throughout their interaction with the system while maintaining a clear record of activity for compliance and auditing purposes.

In some embodiments, the display module 216 is configured to display one or more graphic user interfaces, including, e.g., one or more user interfaces, one or more consumer interfaces, one or more video presenter interfaces, etc. In some embodiments, the display module 216 is configured to temporarily generate and display various pieces of information in response to one or more commands or operations. The various pieces of information or data generated and displayed may be transiently generated and displayed, and the displayed content in the display module 216 may be refreshed and replaced with different content upon the receipt of different commands or operations in some embodiments. In such embodiments, the various pieces of information generated and displayed in a display module 216 may not be persistently stored. The display module 216 provides alerts to the user device which can be viewed and acknowledged by the user.

The display module 216 is responsible for rendering medical record data in a clear and visually structured format. It works in tandem with the user interface module 218 to translate backend data structures into graphical and textual components that users can interpret easily. This includes tables of lab results, medication timelines, charts of vitals, and clinical note summaries.

The display module supports adaptive rendering, allowing the content to scale and reorganize based on device type, screen size, and user accessibility preferences. The module may also include conditional formatting rules, such as color-coding abnormal results or flagging overdue procedures, enhancing situational awareness and data comprehension. This visualization capability ensures that users can quickly understand key information without needing to interpret raw data.

The user interface (UI) module 218 is the main interaction layer between the user and the system. It comprises all graphical elements, interactive buttons, input forms, and layout templates presented through the client application. Designed for both web and mobile platforms, the UI module supports responsive design and adheres to accessibility standards such as WCAG 2.1.

In some embodiments, the module allows customization based on user preferences stored in the user module 212. It provides intuitive navigation pathways to access different types of medical records, filter views, and generate downloadable reports. The UI module is tightly integrated with backend modules to reflect real-time data and alert updates dynamically.

The authentication and security module 220 provides identity verification, session control, and data protection mechanisms. It supports multi-factor authentication (MFA), biometric login, and secure token exchange. This module is responsible for ensuring that only authorized users can view, edit, or share medical records stored in the system.

It also oversees encryption of data at rest and in transit using standards such as AES-256 and TLS 1.3. Additionally, this module manages audit logs of user activity, which are essential for detecting unauthorized access and ensuring regulatory compliance. In some configurations, the module can trigger alerts for suspicious behavior or login attempts from unknown devices.

The data aggregation module 222 collects and consolidates medical records from multiple external sources into a unified internal format. These sources may include EHRs, diagnostic labs, imaging centers, and pharmacy systems. The module uses data adapters or connectors that are customized to interface with source-specific APIs, HL7 feeds, or custom file structures.

This module ensures deduplication and consistency of patient identifiers across incoming datasets. It is capable of resolving conflicting values and merging longitudinal records from different facilities. Aggregated data is routed to the data normalization module 228 for further transformation and standardization before it is displayed or stored.

The medical record storage module 224 serves as the primary repository for all patient health data. It is optimized for secure, scalable storage of large volumes of structured and unstructured medical records, including lab results, imaging metadata, discharge summaries, and prescriptions. The storage module supports time-series organization and tagging by data source and document type.

In some embodiments, the module utilizes sharing or partitioning strategies to ensure performance under high query loads. The system can also replicate critical data across geographically distributed nodes to maintain availability during network or infrastructure disruptions. Secure access control is enforced through permissions defined in the authentication module.

The synchronization module 226 automates the retrieval of new or updated medical data from external systems at scheduled intervals. It may also be triggered by specific events, such as the completion of a clinical encounter or the upload of a new lab result. This ensures that the user always has access to the most current version of their medical information.

The module includes error detection and retry logic to handle communication failures and prevent data loss. Additionally, it maintains a synchronization log that records the status of each data pull, timestamp, and any encountered discrepancies. This audit trail is essential for debugging and ensuring data completeness.

The data normalization module 228 standardizes incoming medical data into a consistent schema that conforms to recognized formats such as HL7, FHIR, or custom-defined templates. This allows records from disparate sources to be presented in a uniform structure within the application.

Normalization includes unit conversion, terminology mapping (e.g., SNOMED, LOINC), and correction of formatting inconsistencies. This module improves the interoperability of the system and simplifies data presentation, analysis, and sharing. It also allows downstream modules like analytics and visualization to process data efficiently without needing to interpret source-specific quirks.

The notification module 230 generates system alerts, reminders, and user-specific notifications regarding new data entries, abnormal results, or pending user actions. Notifications may be sent via email, push notification, or displayed within the application interface.

Users can configure preferences such as notification type, delivery method, and threshold sensitivity. For example, users may choose to receive alerts only for critical lab values or upcoming appointments. This module is essential for improving patient engagement and promoting proactive health management.

The QA testing and framework module 234 manages quality assurance workflows for validating the application's performance, security, and user experience. It supports both automated and manual testing methodologies including unit tests, integration tests, and regression testing.

Test cases are version-controlled and linked to development milestones, ensuring traceability of changes and improvements. This module also logs test coverage metrics and generates reports for compliance and internal review. A robust QA framework reduces deployment risk and ensures high system reliability across updates.

The API integration module 236 allows third-party systems to interface with the application via RESTful APIs, GraphQL queries, or other standardized protocols. It includes authentication gateways, version control mechanisms, and access rate limitations to manage external traffic.

This module enables secure and scalable communication with labs, pharmacies, insurance platforms, and wearable device APIs. It also supports bidirectional data exchange, allowing external systems to write back updates or receive structured health summaries from the platform. The integration layer is essential for building an ecosystem around the application.

The analytics and visualization module 238 processes health data to identify trends, patterns, and anomalies. It supports visual components such as charts, heatmaps, and timelines to help users track their health over time.

The module may offer predictive tools, such as forecasting future lab value trends or identifying health risk indicators. Insights can be exported or shared with providers to aid in treatment planning. The visual interface is user-friendly and supports multiple formats for accessibility.

The code and version management module 240 tracks software changes across all components of the system. It interfaces with version control repositories such as Git to maintain history, branches, and deployment tags.

This module ensures reproducibility, enables rollbacks in case of deployment errors, and supports collaborative development. It is also used to document changes made during feature updates, bug fixes, or infrastructure migrations. Version control is essential for maintaining system integrity and managing compliance.

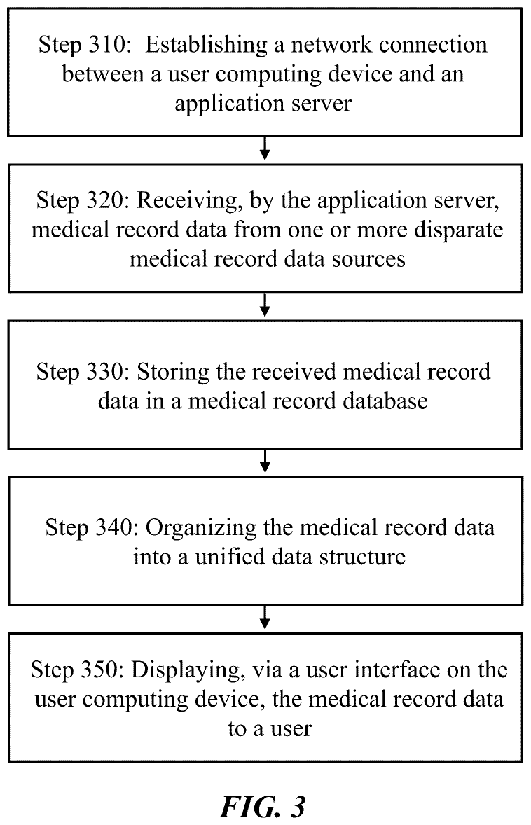

FIG. 3 illustrates a flowchart of a method for accessing and managing medical record data. At step 310, a secure network connection is established between a user computing device and the application server. The user device may be a smartphone, tablet, or computer configured to access the system via a web browser or native application. This connection is established using standard internet protocols over HTTPS with SSL/TLS encryption to ensure secure transmission.

During this phase, the system may invoke an authentication routine to verify the user's credentials, using a username and password combination, multi-factor authentication, or biometric login. Once authenticated, a session token is issued to authorize continued communication between the device and server. This secure communication layer ensures that only validated users are granted access to sensitive health data throughout the session.

In step 320, following authentication and connection, the application server retrieves medical record data from one or more disparate medical data sources. These sources may include electronic health record (EHR) systems, diagnostic laboratories, imaging centers, pharmacies, and insurance platforms. Data may be retrieved via RESTful APIs, HL7 feeds, custom XML endpoints, or secure file exchanges.

To optimize data acquisition, the system may use a polling mechanism or event-driven triggers to initiate requests. The retrieved data may include structured records such as lab results or prescriptions, as well as unstructured documents like clinical notes or imaging summaries. This step is handled by the communication module and the data aggregation module, which work together to ingest and prepare the data for integration into the system.

In step 330, once received, the medical data is stored in a secure, centralized medical record database managed by the storage engine. The data is first parsed and validated for completeness, format compliance, and integrity. Any malformed or incomplete records may be flagged for administrator review or user notification. Valid data is indexed and stored using a schema that supports fast retrieval by patient ID, date, source, and record type.

The medical record storage module also ensures that data is encrypted at rest using advanced encryption standards such as AES-256. In some embodiments, the system supports distributed storage with automated backups and geographic replication to ensure data availability and disaster recovery. Storing the data centrally allows the user to view all relevant records in one interface, regardless of source origin.

In step 340, the medical data is organized into a unified data structure. This structure normalizes and harmonizes incoming records using standard taxonomies such as SNOMED CT, LOINC, or ICD-10. The data normalization module ensures that clinical values are consistently represented, units of measure are converted appropriately, and timestamps are synchronized across time zones.

This step also involves categorizing the data by clinical type (e.g., lab results, medications, imaging) and linking related entries across visits or providers. By transforming disparate records into a cohesive structure, the system supports meaningful longitudinal views and enables accurate filtering, sorting, and searching. This unified data model also facilitates further analysis by visualization or analytics engines.

In step 350, the organized and normalized medical data is displayed to the user through a dynamic user interface. This interface is generated by the user interface module and rendered according to the device's screen size, orientation, and accessibility settings. Users can interact with records by expanding views, applying filters, downloading specific documents, or sharing selected content.

The display interface may include visual aids such as color-coded flags for abnormal results, timelines for medication adherence, and summary charts for vitals or test trends. Notifications of new records or urgent updates may also be presented at this stage. By providing users with clear, actionable, and accessible insights into their medical data, the system empowers more informed health decisions and supports proactive management of care.

The described system provides a practical computing architecture for collecting, processing, and displaying medical record data across multiple sources. Unlike conventional systems that passively store user-input records, the platform described herein includes active communication and data normalization mechanisms that enable seamless integration from electronic health records, diagnostic labs, and other third-party data sources. These interactions are managed through purpose-built modules configured to improve data acquisition, accuracy, and real-time accessibility.

The communication module 202 and data aggregation module 222 work together to manage incoming record requests from various healthcare data systems. These modules are configured with data source adapters, which allow secure, authenticated communication over standard protocols and enable retrieval of structured or unstructured health data. Once received, the records are parsed and formatted into an intermediate structure, which is then processed by subsequent modules within the system architecture.

One notable feature is the system's ability to reorganize and harmonize disparate records into a unified data structure. This task is handled by the data normalization module 228, which employs logic for value standardization, terminology mapping, and structural formatting. By translating data into a consistent schema, the system enhances interoperability and enables responsive search, sorting, and filtering functions that would not be possible using unprocessed or fragmented inputs.

The user interface module 218 and display module 216 are configured to provide an interactive environment through which users can navigate longitudinal health histories, monitor abnormal values, and view summaries of clinical events. The interface includes condition-based formatting, expandable panels, and graphically encoded indicators such as timelines and charts. These features allow for dynamic presentation of data based on source type, timestamp, or medical category, improving usability and data comprehension.

To ensure continuous access to up-to-date health data, the system includes a synchronization module 226 configured to monitor known data sources and pull new or updated records on a scheduled or event-driven basis. This synchronization occurs in the background, without requiring user input, and updates the system database with timestamped entries while maintaining a synchronization log. Such functionality ensures that the records presented to the user are as current as possible while reducing the administrative burden on the end-user.

Security is enforced by the authentication and security module 220, which manages identity verification, access rules, and encryption protocols. This module may support a range of user authentication mechanisms, including passwords, device-based authentication tokens, and biometric inputs. It also governs the permissions associated with record viewing, sharing, and export, ensuring that sensitive information is protected throughout all operations conducted within the system.

When users elect to export or share medical records, the system utilizes the export and sharing module 232, which formats data into machine-readable or human-readable formats, such as HL7 bundles, FHIR-compliant JSON, or PDF summaries. The module may also include functionality for generating secure access links with user-defined time limits and data access scopes. These operations enable users to transfer their records to external providers or care teams efficiently, while retaining control over access conditions and record integrity.

In one alternative embodiment, the system is implemented using a special-purpose embedded computing device integrated into a clinical environment, such as a kiosk located in a hospital or pharmacy. This device includes pre-installed software that connects directly to institutional medical records and prints summarized health information for walk-in users.

In another embodiment, the system is configured for offline operation with delayed synchronization, wherein patient data collected through mobile applications is temporarily stored in a local encrypted cache. Once network connectivity is reestablished, the synchronization module transmits the stored data to the central database.

The system may be deployed within a federated data architecture, where no single central database exists. Instead, each healthcare provider maintains their own node within a distributed ledger (such as a permissioned blockchain), and the user device queries multiple nodes to assemble a real-time patient record.

In some embodiments, the system may include real-time biometric authentication using facial recognition or fingerprint scanning to unlock specific sections of the medical data. This feature requires integration with device-level biometric sensors and leverages secure enclave technologies available in modern smartphones.

In some versions of the system, the user interface module dynamically adjusts based on machine learning-based health insights. For example, a user's recent test results may trigger the system to prioritize related health information or suggest relevant readings and follow-ups. The use of machine learning introduces a non-conventional, predictive engine that adapts system behavior in real-time.

In a hybrid embodiment designed for enterprise use, the system is installed within a hospital intranet, allowing integration with on-premises EHR systems without exposing patient data to the public internet. This localized deployment includes secure data bridges and virtual private network (VPN) tunnels to enable compliance with institutional firewalls and legacy system interfaces. The system's adaptability to this closed-network environment makes it distinct from conventional cloud-based health portals, strengthening novelty and non-obviousness claims.

In some implementations, the system is integrated with clinical decision support (CDS) tools, where the application not only displays records but also cross-references them against a medical knowledge base. When certain data patterns are detected, such as potential contraindications or unfulfilled diagnostic criteria, the system presents real-time prompts to the user. These alerts provide clinical value and represent a technical improvement over generic data repositories.

To reinforce structural specificity, an embodiment of the system includes a hardware-based security token (e.g., a FIDO-compliant USB device or NFC card) required for system access. This prevents unauthorized logins and adds a cryptographic identity verification layer that operates independently of software credentials. The inclusion of dedicated hardware transforms the system from a purely software-driven process into one that is securely tied to physical elements.

Lastly, the invention may be integrated into a patient-controlled medical data wallet, where the user can assign time-limited access rights to selected providers. These rights may include smart contracts that automatically revoke access after a predefined condition is met, such as a treatment session ending or a deadline passing. This embodiment shows user-directed, rule-based control over data sharing, combining technical specificity with user empowerment, which supports arguments against both abstractness and obviousness.

In this disclosure, the subject matter has been described in the general context of computer-executable instructions of a computer program product running on a computer or computers, and those skilled in the art would recognize that this disclosure can be implemented in combination with other program modules. Generally, program modules include routines, programs, components, data structures, etc. that perform particular tasks and/or implement particular abstract data types. Those skilled in the art would appreciate that the computer-implemented methods disclosed herein can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated embodiments can be practiced in distributed computing environments where tasks are performed by remote processing devices that are linked through a communications network. Some embodiments of this disclosure can be practiced on a stand-alone computer. In a distributed computing environment, program modules can be located in both local and remote memory storage devices.

In this disclosure, the terms “component,” “system,” “platform,” “interface,” and the like, can refer to and/or include a computer-related entity or an entity related to an operational machine with one or more specific functionalities. The disclosed entities can be hardware, a combination of hardware and software, software, or software in execution. For example, a component can be a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As another example, a component can be an apparatus that provides specific functionality through electronic components without mechanical parts, wherein the electronic components can include a processor or other means to execute software or firmware that confers at least in part the functionality of the electronic components. In some embodiments, a component can emulate an electronic component via a virtual machine, e.g., within a cloud computing system.

The phrase “application” as is used herein means software other than the operating system, such as Word processors, database managers, Internet browsers and the like. Each application generally has its own user interface, which allows a user to interact with a particular program. The user interface for most operating systems and applications is a graphical user interface (GUI), which uses graphical screen elements, such as windows (which are used to separate the screen into distinct work areas), icons (which are small images that represent computer resources, such as files), pull-down menus (which give a user a list of options), scroll bars (which allow a user to move up and down a window) and buttons (which can be “pushed” with a click of a mouse). A wide variety of applications is known to those in the art.

The phrases “Application Program Interface” and API as are used herein mean a set of commands, functions and/or protocols that computer programmers can use when building software for a specific operating system. The API allows programmers to use predefined functions to interact with an operating system, instead of writing them from scratch. Common computer operating systems, including Windows, Unix, and the Mac OS, usually provide an API for programmers. An API is also used by hardware devices that run software programs. The API generally makes a programmer's job easier, and it also benefits the end user since it generally ensures that all programs using the same API will have a similar user interface.

The phrase “central processing unit” as is used herein means a computer hardware component that executes individual commands of a computer software program. It reads program instructions from a main or secondary memory, and then executes the instructions one at a time until the program ends. During execution, the program may display information to an output device such as a monitor.

The term “execute” as is used herein in connection with a computer, console, server system or the like means to run, use, operate or carry out an instruction, code, software, program and/or the like.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

What is claimed is:

1. A system for accessing and managing medical record data, the system comprising:
at least one user computing device in operable connection with a user network;
an application server in operable communication with the user network, the application server configured to host an application program for collecting, accessing, and managing medical record data;
a medical record database configured to receive a plurality of medical record data from one or more disparate medical record data sources; and
a user interface provided by the application program, the user interface configured to display the plurality of medical record data to the user.

2. The system of claim 1, wherein the application program is configured to authenticate the user before allowing access to the medical record data.

3. The system of claim 1, wherein the user interface allows the user to download the medical record data in a standard file format.

4. The system of claim 1, wherein the application server is further configured to perform periodic synchronization with the disparate medical record data sources.

5. The system of claim 1, wherein the medical record data comprises laboratory results, imaging reports, prescriptions, and clinician notes.

6. The system of claim 1, wherein the user computing device comprises a smartphone, tablet, or laptop computer.

7. The system of claim 1, wherein the application program includes an alert system for notifying users of new or updated medical records.

8. The system of claim 1, wherein the medical record database is cloud-based and accessible via an application programming interface (API).

9. A method for accessing and managing medical record data, the method comprising the steps of:
establishing a network connection between a user computing device and an application server;
receiving, by the application server, medical record data from one or more disparate medical record data sources;
storing the received medical record data in a medical record database;
organizing the medical record data into a unified data structure; and
displaying, via a user interface on the user computing device, the medical record data to a user.

10. The method of claim 9, further comprising authenticating the user via a secure login process before providing access to the medical record data.

11. The method of claim 9, wherein the step of receiving medical record data includes querying an external health information exchange system.

12. The method of claim 9, further comprising encrypting the medical record data prior to storing it in the medical record database.

13. The method of claim 9, further comprising categorizing the medical record data based on source type and timestamp.

14. The method of claim 9, wherein the displaying step includes generating visual summaries of the data such as charts or timelines.

15. The method of claim 9, wherein the unified data structure is compliant with HL7 or FHIR standards.

16. A non-transitory computer-readable medium storing instructions that, when executed by one or more processors, cause a computing device to perform operations comprising:
receiving a request for medical record data from a user;
retrieving medical record data from a plurality of disparate medical record data sources;
storing the retrieved data in a medical record database;
integrating the retrieved data into a unified format; and
transmitting the integrated data to a user interface associated with the user computing device for display.
displaying, via a user interface on the user computing device, the medical record data to a user.

17. The non-transitory computer-readable medium of claim 16, wherein the operations further comprise authenticating the identity of the user prior to retrieving the medical record data.

18. The non-transitory computer-readable medium of claim 16, wherein the operations further comprise filtering the retrieved data based on user-defined preferences.

19. The non-transitory computer-readable medium of claim 16, wherein the operations further comprise generating an exportable file for patient-controlled sharing with third parties.

20. The non-transitory computer-readable medium of claim 16, wherein the operations further comprise storing a log of data retrieval events for auditing purposes.
